(19) United States
(12) Patent Application Publication
(10) Pub. No.: US 2025/0244326 A1
(43) Pub. Date: Jul. 31, 2025

(54) BLOOD BIOMARKERS IN LONG-COVID-19

(71) Applicant: LONDON HEALTH SCIENCES CENTRE RESEARCH INC., London (CA)

(72) Inventor: Douglas D. Fraser, London (CA)

(73) Assignee: LONDON HEALTH SCIENCES CENTRE RESEARCH INC., London, ON (CA)

(21) Appl. No.: 18/833,601

(22) PCT Filed: Feb. 3, 2023
(86) PCT No.: PCT/CA2023/050145

§ 371 Date: Jul. 26, 2024

Related U.S. Application Data
(60) Provisional application No. 63/306,848, filed on Feb. 4, 2022.

(51) Int. Cl. G01N 33/569; A61K 38/17; A61K 38/18; A61K 38/48; G16B 40/00

(52) U.S. Cl. CPC G01N 33/56983; A61K 38/178; A61K 38/1891; A61K 38/4886; G16B 40/00; G01N 2333/165; G01N 2333/475; G01N 2333/515; G01N 2333/70503; G01N 2333/70525; G01N 2333/70546; G01N 2333/70564; G01N 2333/71; G01N 2333/7452; G01N 2333/96494

(57) ABSTRACT

A method of diagnosing long-COVID in a subject, the method comprising: (a) measuring the level of one or more biomarkers in a test sample obtained from the subject, and (b) comparing the level of the one or more biomarkers in the test sample with a healthy control and/or an acute COVID-19 reference level value of said one or more biomarkers, wherein an increase in the level of the one or more biomarkers in the test sample relative to the healthy control and/or acute COVID-19 reference level value of said one or more biomarkers is indicative of long-COVID diagnosis, and wherein the one or more biomarkers are selected from Table 5. In one embodiment, the one or more biomarkers include ANG-1, P-Sel and MMP-1. A method of treating long-COVID comprising administering to a subject one or more of the biomarkers are selected from Table 5.

### CROSS REFERENCE TO RELATED APPLICATIONS

This application is a national stage application under 35 U.S.C. 371 of International Application No. PCT/CA2023/050145, filed Feb. 3, 2023, which in turn claims the benefit under 35 U.S.C. 119(e) of U.S. Provisional Ser. No. 63/306,848, filed Feb. 4, 2022, the contents of each of which are hereby incorporated by reference into the present disclosure.

### FIELD

This disclosure relates to blood biomarkers in long-COVID and to methods of diagnosis of long-COVID using said blood biomarkers.

### BACKGROUND

Coronavirus disease 2019 (COVID-19) is caused by the highly transmissible severe acute respiratory syndrome coronavirus 2 (SARS-CoV-2) (1). SARS-CoV-2 binds the cell surface angiotensin-converting enzyme 2 (ACE2) receptor, resulting in cell entry and viral replication (2). An innate immune response follows SARS-CoV-2 infection (3) and includes increased interferons, tumor necrosis factor, bradykinin, serine proteases, soluble thrombomodulin and clot lysis times (4-8), all contributing to microvascular and thrombotic disease (9, 10). COVID-19 induces a wide range of disease severity, with hospitalized patients suffering an overall mortality rate of approximately 27% (11).

Survivors of COVID-19 often suffer diffuse symptoms that can persist for 2-7 months, referred to as “Long-COVID” (12-15). Several mechanisms have been proposed to explain the diffuse symptoms associated with Long-COVID, including the organ-specific expression of ACE-2 receptors that may predispose particular systems to greater tissue injury and prolonged healing (e.g., lung, heart, brain) (16), and/or microvascular endothelial dysfunction secondary to exacerbated inflammation and thrombotic mechanisms (9, 17). A lack of pathophysiological mechanisms and specific diagnostic markers has led some to question whether Long-COVID is a true disease entity (18). Given the ongoing debate associated with Long-COVID diagnoses, and the fact that only symptomatic treatments are available, two major priorities to optimize care of Long-COVID patients include early disease recognition with specific diagnostics, as well as identification of molecular mechanisms for future targeted therapies.

Accurate diagnosis and targeted therapies for LONG-COVID are lacking, making the identification of biomarkers and pathophysiological mechanisms critical to optimize Long-COVID care.

### SUMMARY

In one embodiment, the present disclosure is a method of diagnosing long-COVID in a subject, the method comprising: (a) measuring the level of one or more biomarkers in a test sample obtained from the subject, and (b) comparing the level of the one or more biomarkers in the test sample with a healthy control and/or an acute COVID-19 reference level value of said one or more biomarkers, wherein an increase in the level of the one or more biomarkers in the test sample relative to the healthy control and/or acute COVID-19 reference level value of said one or more biomarkers is indicative of long-COVID diagnosis, and wherein the one or more biomarkers are selected from Table 5.

In one embodiment of the method of diagnosing long-COVID, the one or more biomarkers include one or more of ANG-1, P-Sel and MMP-1.

In another embodiment of the method of diagnosing long-COVID, the one or more biomarkers is ANG-1. P-Sel or MMP-1.

In another embodiment of the method of diagnosing long-COVID, the one or more biomarkers are ANG-1 and P-Sel.

In another embodiment of the method of diagnosing long-COVID, the one or more biomarkers are ANG-1 and MMP-1.

In another embodiment of the method of diagnosing long-COVID, the one or more biomarkers are P-Sel and MMP-1.

In another embodiment of the method of diagnosing long-COVID, the one or more assays is a proteomic assay.

In another embodiment of the method of diagnosing long-COVID, when the subject is diagnosed with long-COVID, the method further includes treating the subject with a long-COVID therapy.

In another embodiment of the method of diagnosing long-COVID, the therapy includes one or more interventions listed in Table 1.

In another embodiment of the method of diagnosing long-COVID, the therapy comprises administering to the subject a treatment that promotes angiogenesis.

In another embodiment of the method of diagnosing long-COVID, the therapy comprises administering to the subject accelerators of angiogenesis.

In another embodiment of the method of diagnosing long-COVID, the therapy comprises administering to the subject one or more of the biomarkers listed in Table 5.

In another embodiment of the method of diagnosing long-COVID, the therapy comprises administering to the subject one or more of ANG-1, P-Sel and MMP-1.

In another embodiment of the method of diagnosing long-COVID, step (b) is done using machine learning.

In another embodiment, the present disclosure provides for a method of diagnosing long-COVID in a subject, the method comprising: (a) using machine learning to compare the level of one or more biomarkers in a test sample obtained from the subject with healthy control and/or acute COVID-19 reference level value of said one or more biomarkers, wherein the one or more biomarkers are selected from ANG-1, P-Sel, MMP-1, VE-Cad, Syn-1, Endoglin, PECAM, VEGF-A, ICAM-1, VLA-4, E-Sel, Thrombomodulin, VEGF-R2, and VEGF-R-3, and (b) determining whether the subject is positive or negative for long-COVID based on said comparison.

Another embodiment provides for a method of treating long-COVID in a subject, the method comprising administering to the subject one or more of the biomarkers listed in Table 5. In aspects, the one or more biomarkers include ANG-1, P-Sel and MMP-1.

In another embodiment, the present disclosure relates to a method of treating long-COVID in a subject, the method comprising providing a subject having an increase in the level of one or more biomarkers selected from Table 5 relative to a healthy control and/or acute COVID-19 reference level value of said one or more biomarkers, and (b) treating the subject with a therapy effective against long-COVID.

In one embodiment of the method of treating long-COVID in a subject, the therapy includes one or more interventions listed in Table 1.

In another embodiment of the method of treating long-COVID in a subject, said therapy comprises administering to the subject a treatment that promotes angiogenesis.

In another embodiment of the method of treating long-COVID in a subject, said therapy comprises administering to the subject accelerators of angiogenesis.

In another embodiment of the method of treating long-COVID in a subject, said therapy comprises administering to the subject one or more of the biomarkers listed in Table 5.

In another embodiment of the method of treating long-COVID in a subject, said therapy comprises administering to the patient one or more of ANG-1, P-Sel and MMP-1.

In another embodiment, the present disclosure relates to a use of one or more of the biomarkers listed in Table 5 for the treatment of long-COVID in a subject.

In one embodiment of the use of one or more of the biomarkers listed in Table 5 for the treatment of long-COVID in a subject, the one or more biomarkers include one or more of ANG-1, P-Sel and MMP-1.

In another embodiment, the present disclosure relates to a use of one or more of the biomarkers listed in Table 5 for the manufacture of a medicament for treating long-COVID in a subject.

In one embodiment of the use of one or more of the biomarkers listed in Table 5 for the manufacture of a medicament for treating long-COVID in a subject, the one or more biomarkers include one or more of ANG-1, P-Sel and MMP-1.

### BRIEF DESCRIPTION OF THE DRAWINGS

The following figures illustrate various aspects and preferred and alternative embodiments of this disclosure.

FIGS. 1A-1B. Identification of important vascular transformation blood biomarkers in Long-COVID outpatients. 1A) Subjects plotted in two-dimensions, following tSNE dimensionality reduction of all fourteen significant biomarkers, shows separation cluster of Long-COVID outpatients with some mixing with acutely ill COVID-19 inpatients and healthy control subjects. 1B) Subjects plotted in two-dimensions, following tSNE dimensionality reduction of three selected biomarkers, ANG-1, P-SEL and MMP-1, showed distinct separation and clustering of Long-COVID outpatients from acutely ill COVID-19 inpatients and healthy control subjects.

FIG. 2. A heatmap demonstrated the pairwise Eucladian Distance between cohort's biomarker profiles with respect to ANG-1, P-SEL and MMP-1. Lower distances between patients indicate similar biomarker profiles while larger distances indicate large differences between profiles (distance was pseudocolored on the bar scale). The biomarker profile of Long-COVID outpatients is distinctively different from all other cohorts. The heatmap color scale was capped at 500,000 to restrict interpretation bias from Long-COVID outliers (max value 1,200,000) and allow for more visible details.

FIGS. 3A-3F. Box plots and receiver operating characteristic (ROC) curves for the three leading vascular transformation blood biomarkers, ANG-1, P-SEL and MMP-1. 3A) A boxplot demonstrating significantly elevated blood ANG-1 concentrations in Long-COVID outpatients (****P<0.0001). 3B) ROC curves demonstrating the excellent Long-COVID classification potential of blood ANG-1 versus healthy control subjects (AUC=1.00, P<0.0001) and acutely ill COVID-19 inpatients (AUC=1.00, P<0.0001). 3C) A boxplot demonstrating significantly elevated blood P-SEL concentrations in Long-COVID outpatients (****P<0.0001; ***P<0.001). 3D) ROC curves demonstrating the excellent Long-COVID classification potential of blood P-SEL versus healthy control subjects (AUC=1.00, P<0.0001) and acutely ill COVID-19 inpatients (AUC=1.00, P<0.0001). 3E) A boxplot demonstrating significantly elevated blood MMP-1 concentrations in Long-COVID outpatients (****P<0.0001; ***P<0.001; *P<0.05). 3F) ROC curves demonstrating the excellent Long-COVID classification potential of blood MMP-1 versus healthy control subjects (AUC=1.00, P<0.0001) and acutely ill COVID-19 inpatients (AUC=0.94, P<0.0001).

FIGS. 4A-4D. Violin plots demonstrating the distribution of ANG-1 and MMP-1 blood concentration in Long-COVID patients relative to sex and interventions at follow-up. 4A) A violin plot demonstrating significantly elevated concentration of blood ANG-1 in Long-COVID female outpatients (*P<0.05). 4B) A violin plot demonstrating significantly elevated concentration of blood ANG-1 in Long-COVID outpatients that had no interventions at follow-up (*P<0.05). 4C) A violin plot demonstrating significantly elevated concentration of blood MMP-1 in Long-COVID female outpatients (*P<0.05). 4D) A violin plot demonstrating significantly elevated concentration of blood MMP-1 in Long-COVID outpatients that had no interventions at follow-up (*P<0.05). Given the limited number of patients within this subgroup, the data was not corrected for multiple comparisons and should be considered exploratory.

### DESCRIPTION

### Abbreviations

Unless defined otherwise, all technical and scientific terms used herein have the same meaning as commonly understood by one of ordinary skill in the art to which this disclosure belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this disclosure.

The contents of all documents (including patent documents and non-patent literature) cited in this application are incorporated herein by reference.

All numerical designations, e.g., levels, amounts and concentrations, including ranges, are approximations that typically may be varied (+) or (−) by increments of 0.1, 1.0, or 10.0, as appropriate. All numerical designations may be understood as preceded by the term “about”.

“COVID-19—subjects” or “COVID-19 negative subjects” are subjects who are septic with Acute lung injury (ALI), but are confirmed SARS-CoV-2 negative.

“COVID-19+ subjects” (or patients) or “COVID-19 subjects” are subjects who are confirmed SARS-COVID-19 positive. In the Examples, the studies have been carried out with ward COVID-19+ subjects (mild/moderate disease) and with intensive care unit COVID-19+ subjects (severe disease or critically ill).

The term “subject” as used herein refers all members of the animal kingdom including mammals, preferably humans.

The term “patient” as used herein refers to a subject that is COVID-19+.

“Plasma” is the clear, straw-colored liquid portion of blood that remains after red blood cells, white blood cells, platelets and other cellular components are removed.

The term “pharmaceutically acceptable carrier”, “pharmaceutically acceptable excipient”, “physiologically acceptable carrier”, or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

Angiogenesis is a multistep process for the formation of new blood vessels. “Vaso-proliferative proteins” or “angiogenic proteins” refer to proteins that lead to activation of the cellular pathways that result in angiogenesis. Proteins, including angiogenic proteins, can be measured with antibody tests (i.e., Western blotting, Luminex bead-based assays, Proximity Extension Assay (PEA), planar multiplex assays, lateral flow assays, electrical conductivity devices, electrochemiluminescence, proximal extension assay with oligonucleotide-labeled antibodies, ELISA and RIA), flow cytometry or mass spec techniques. Enzymes can be measured with enzyme assays that measure either the consumption of a substrate or production of product over time. Differential expression profiles may have important diagnostic value, even in the absence of specifically identified proteins. Single protein spots can then be detected, for example, by immunoblotting, multiple spots or proteins using protein microarrays. The term “proteomic profile” is used to refer to a representation of the expression pattern of a plurality of proteins in a biological sample, e.g., a biological fluid at a given time. The proteomic profile can, for example, be represented as a mass spectrum, but other representations based on any physicochemical or biochemical properties of the proteins are also included. Thus, the proteomic profile may, for example, be based on differences in the electrophoretic properties of proteins, as determined by two-dimensional gel electrophoresis, e.g., by 2-D PAGE, and can be represented, e.g., as a plurality of spots in a two-dimensional electrophoresis gel. The proteomic profile typically represents or contains information that could range from a few peaks to a complex profile representing 50, 1,000 or more peaks. Thus, for example, the proteomic profile may contain or represent at least 2, or at least 5 or at least 10 or at least 15, or at least 20, or at least 25, or at least 30, or at least 35, or at least 40, or at least 45, or at least 50 proteins, or over 1,000 proteins.

Any suitable point-of-care measurement devices can be used to measure the proteins of the present disclosure, including testing with portable, table/counter-top, hand-held, lateral flow device (including lateral flow immunochromatographic assay), chip or MS protein testing instruments.

The terms “active ingredient”, “active compound”, and “active substance” refer to a compound, which is administered, alone or in combination with one or more pharmaceutically acceptable excipients or carriers, to a subject for treating, preventing, or ameliorating one or more symptoms of COVID19 pathology.

The terms “agent”, “drug”, “therapeutic agent”, and “chemotherapeutic agent” refer to a compound, or a pharmaceutical composition thereof, which is administered to a subject for treating, preventing, or ameliorating one or more symptoms of COVID19 pathology.

“Long-COVID” refers to subjects not recovering for several weeks or months following the start of symptoms that were suggestive of COVID-19 and to survivors of COVID-19 that suffer diffuse symptoms that can persist for at least two months. According to the CDC website “Many post-COVID conditions can be improved through already established symptom management approaches (e.g., breathing exercises to improve symptoms of dyspnea). Creating a comprehensive rehabilitation plan may be helpful for some patients and might include physical and occupational therapy, speech and language therapy, vocational therapy, as well as neurologic rehabilitation for cognitive symptoms. A conservative physical rehabilitation plan might be indicated for some patients (e.g., persons with post-exertional malaise); consultation with physiatry for cautious initiation of exercise and recommendations about pacing may be useful. Gradual return to exercise as tolerated could be helpful for most patients. Optimizing management of underlying medical conditions might include counseling on lifestyle components such as nutrition, sleep, and stress reduction (e.g., meditation).” [Taken from CDC website: https://www.cdc.gov/coronavirus/2019-ncov/hcp/clinical-care/post-covid-management.html.]

The compositions comprising one or more of the biomarkers of Table 3, 5 of the present disclosure include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration. The compositions may conveniently be presented in unit dosage form and may be prepared by any of the methods well known in the art of pharmacy.

Formulations of the compounds of Table 5 suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

In addition to the formulations described previously, the compounds may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

The amount of active ingredient that may be combined with the carrier materials to produce a single dosage form will vary depending upon the host treated and the particular mode of administration.

The compounds can be administered in various modes, e.g., orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

The present disclosure relates to the diagnosis of long-COVID using one or more of the biomarkers listed in Table 5. In one embodiment, the biomarkers include one or more of ANG-1, P-Sel, MMP-1 and any combination thereof. Also, methods of treating long-COVID in a patient comprising administering to the patient one or more of the biomarkers listed in Table 4. In one embodiment of the method to treat long-COVID, the biomarkers include one or more of ANG-1, P-Sel, MMP-1 and any combination thereof. Depending on the symptoms, treatment of long-COVID can include any one or more of the interventions listed in Table 1. These standard symptomatic treatments for Long-COVID includes therapeutic exercise, physiotherapy, oxygen, inhalers (salbutamol, steroids), nasal sprays and/or anticoagulation. In one embodiment, treatment of long-COVID includes target therapy comprising administering to the patient one or more of the biomarkers listed in Table 5. In another embodiment, treatment of long-COVID includes administering to the patient an accelerator of angiogenesis to encourage the angiogenesis process to be faster and complete.

### Therapeutic Angiogenesis

1. Therapeutic Angiogenic Drugs (Li V W, Kung E F, Li W W. Molecular Therapy for Wounds: Modalities for stimulating Angiogenesis and Granulation. Manual of Wound Management (Bok Lec, Editor) McGraw Hill, 2004, p. 17-43; Li W, Talcott K, Zhai A, Kruger E, Li V. The Role of Therapeutic Angiogenesis in Tissue Repair and Regeneration Adv Skin Wound Care 2005; 18:491-500; Smiell J M, Wieman T J, Steed D L, et al. Efficacy and safety of becaplermin (recombinant human platelet-derived growth factor-BB) in patients with nonhealing, lower extremity diabetic ulcers: a combined analysis of four randomized studies. Wound Repair Regen. 1999; 7:335-346):
Growth factor-based therapies include the only FDA-approved recombinant protein drug recombinant human Platelet-derived growth factor (rhPDGF) (becaplermin, REGRANEX® 0.01% gel), which is indicated for diabetic neuropathic lower extremity ulcers.
Growth factors can also be delivered through autologous isolates of patient platelets such as Autologel, SmartPReP.
Currently, there are no FDA-approved angiogenic drugs for the treatment of ischemic cardiovascular disease.
Some early-stage clinical trials of therapeutic angiogenic agents have demonstrated reductions in symptoms of angina, increase in ability to exercise, and objective evidence of improved perfusion and left ventricular function following therapy.
Therapeutic Angiogenesis Promoting Devices: Negative pressure wound therapy (NPWT) such as the Vacuum Assisted Closure (V.A.C.) system induces angiogenesis through tissue microdeformations and mechanochemical coupling and signal transduction; MIST ultrasound is a low-frequency and low-intensity non-contact device that results in cell stimulation and increased wound perfusion; Hyperbaric Oxygen (HBO) promotes angiogenesis and wound healing by increasing Vascular endothelial growth factor (VEGF) expression and recruiting endothelial progenitor cells.
Cell-Based Therapies:
Tissue engineered products approved by the FDA include the bilayered skin substitute Grafstkin (Apligraf®) and the fibroblast dermal skin substitute Dermagraft. These products contain living or cryopreserved cells on a matrix capable of secreting and releasing multiple angiogenic growth factors into the wound bed.
CD34+ endothelial progenitor cells (EPC) derived from bone marrow or from peripheral blood have been found to enhance angiogenesis in ischemic tissues, increase transcutaneous oxygen, improve ankle-brachial index (ABI), increase collateral vessels by angiography and improve healing of leg ulcers.
Integra® Dermal Regeneration Template is an advanced skin replacement matrix that consists of a complex three-dimensional porous matrix that acts as a scaffold for cell migration and allows for regeneration of the dermal layer of the patient's skin. It can be used for Diabetic Foot Ulcers.

2. VEGF/VPF (Tan, Q., et al., European Journal of Cardio-thoracic Surgery 31 (2007) 806-811; vascular permeability factor (VPF).

3. ANG1/TIE1 pathway—ANG1 has vasculoprotective effects. It enhances the stability of newly formed vessels, inhibits vascular permeability induced by several inflammatory cytokines and attenuates pathological responses, including fibrosis. Recently, the angiopoietin (ANG)-TIE signaling pathway has emerged as an attractive vascular drug target. The ANG-TIE pathway is required for lymphatic and blood vessel development.
1. Recombinant/viral vectors ANG1 (Angiopoietin 1)
2. ANG2 (Angiopoietin 2) inhibitors (ANG2 blocks angiogenesis)

4. Statin therapy
Low-dose statin therapy may promote angiogenesis via multiple mechanisms, including enhanced NO production, augmented VEGF release, and activation of the Akt signaling pathway

5. Therapeutic/Prescribed Exercise
Exercise stimulates angiogenesis in skeletal muscle and heart. A lack of exercise leads to capillary regression.

6. Plasminogen Activator System (Plasmin)
https://www.news-medical.net/health/Angiogenesis-Stimulation.aspx
Plasmin also activates Matrix metalloproteinases (MMPs) such as MMP-1, MMP-3, and MMP-9. These are metalloproteinases.

6. Growth Factors (Fibroblast growth factor 2 (FGF2) and VEGF)
FGF 2 is vital for angiogenesis. It induces multiplication and movement of the cells as well as uPA production by endothelial cells. FGF-2 induces tube formation in collagen gels and alters integrin expression that helps in angiogenesis.

7. Combination therapies—VEGF, FGF, MMP1, plasma, etc. (Sabra, M., et al., Int. J. Mol. Sci. 2021, 22, 3722)

8. Stimulators of angiogenesis: VEGF, FGF, Hepatocyte Growth Factor (HGF), Angiopoietin 1 (Ang1) and Angiopoietin 2 (Ang2), Platelet-derived growth factors (PDGFs), insulin-like growth factor (IGF), Endoglin Interleukin 8, Thyroxin, VE-cadherin, Granulocyte colony-stimulating factor (G-CSF), Integrins, Ephrin, Endothelial nitric oxide synthase (eNOS), Transforming growth factor beta (TGFbeta), YKL40, HIF1α (Hypoxia Inducible Factor 1 Subunit Alpha), HDGF (Heparin Binding Growth Factor), Notch/DLL4 (Delta-like 4), Semaphrorins.

9. Chinese herbal medicines. Chinese herbal medicines that target angiogenesis can provide therapeutic effect, including active components Salvianolic acid A, Tanshinone IIA, Ferulaic acid, Rhodiola, Salidroside, Astragalosides, Berberine, Puerarin and Extract of Geum japonicum. These active components can be obtained from Radix Salvia miltiorrhiza, Radix Angelica Sinensis, Rhizoma Rhodiolae Kirilowii, Shanxi Astragalus membranaceus, Berberis and Berberis aristate, Radix Puerariae, Germ japonicum(Dongqing Guo, et al., Frontiers in Pharmacology, (2018) 9, 428).

In order to aid in the understanding and preparation of the within disclosure, the following illustrative, non-limiting, examples are provided.

### EXAMPLES

### Example 1

### Methods

This study was approved by the Western University, Human Research Ethics Board (HREB): Long-COVID outpatients (HREB #120084, issued Nov. 15, 2021); acutely ill COVID-19 inpatients (HREB #6970, renewed Mar. 17, 2021) and volunteer healthy control subjects (HREB #16986E, renewed Mar. 9, 2021).

### Study Participants and Blood Sampling:

All patients were screened and enrolled from our tertiary care system, The London Health Sciences Centre (London, Ontario, Canada). All patients, both Long-COVID and acutely ill COVID-19, had their COVID-19 status confirmed as part of standard hospital testing by detection of two SARS-CoV-2 viral genes using polymerase chain reaction (19). Long-COVID out patients had been referred to a specialty clinic based on prolonged and diffuse symptoms. Venous blood work was drawn once as part of a larger clinical screen, and excess plasma collected for later research analysis by Pathology and Laboratory Medicine (PaLM). Both Ward and intensive care unit (ICU) patients were enrolled on admission to hospital. Blood sampling for inpatients began on admission, Ward or ICU Day-1 and repeated on Day-3. Daily blood was obtained from critically ill ICU patients via indwelling catheters and if a venipuncture was required, research blood draws were coordinated with a clinically indicated blood draw. In keeping with accepted research phlebotomy protocols for adult patients, blood draws did not exceed maximal volumes (20). Blood was centrifuged and plasma isolated, aliquoted at 250 μL, and frozen at −80° C. All samples remained frozen until use and freeze/thaw cycles were avoided.

The healthy control subjects were individuals without disease, acute illness, or prescription medications that were previously banked in the Translational Research Centre, London, ON (Directed by Dr. D. D. Fraser; https://translationalresearchcentre.com/) (21, 22).

### Patient Demographics, Clinical Data and Cohort Matching

Baseline characteristics for Long-COVID, Ward and, ICU patients were recorded and included age, sex, comorbidities, presenting symptoms, interventions, and laboratory measurements. For Long-COVID patients, we recorded both initial infection variables and clinical variables at follow-up clinic. For the latter, we focused on lingering symptoms, laboratory values and interventions. For ICU patients, we included standard illness severity scores, including Multiple Organ Dysfunction Score (MODS) (23) and Sequential Organ Failure Assessment scores (24). The Pao2 to Fio2 ratio and chest radiograph findings were recorded for all ICU patients. We also recorded clinical interventions received during the observation period including the use of antibiotics, antiviral agents, systemic corticosteroids, vasoactive medications, venous thromboembolism prophylaxis, antiplatelet, or anticoagulation treatment, renal replacement therapy, high flow oxygen therapy, and mechanical ventilation (invasive and non-invasive). Final participant groups were constructed by age- and sex-matching Long-COVID patients with Ward COVID-19 patients, ICU COVID-19 patients and healthy control subjects.

### Multiplex Immunoassay:

Concentrations of vascular transformation blood biomarkers were determined in human plasma using two distinct custom multiplexed immunoassay kits according to manufacturer's instructions (Invitrogen™). The first Endothelial Injury Marker Panel assayed 12 target proteins (no pre-dilution); VEGF-A, VEGF-D, VEGF-R2, VEGF-R3, P-Selectin, E-Selectin, PECAM-1, Angiopoietin-1/ANG-1, MMP-1, Thrombomodulin™, Syndecan-1 (Syn-1) and VLA-4. The second Endothelial Injury Marker Panel assayed 4 target proteins (1:100 pre-dilution); Endoglin, sICAM-1, sVCAM-1 and VE-Cadherin. Both kits utilized Luminex® xMAP™ fluorescent bead-based technology (Luminex Corp., 12212 Technology Blvd, Austin, TX, 78727, USA). The assay plate was treated according to the manufacturer's instructions and quantified on a compatible Luminex® system (Bio-Plex™ 200 system, Bio-Rad Laboratories, 1000 Alfred Nobel Drive, Hercules, CA, 94547, USA).

### Conventional Statistics:

Patient baseline clinical characteristics were reported as Median (IQRs) for continuous variables and frequency (%) for categorical variables. Ward and ICU blood draws from day 1 and day 3 were combined for both groups separately. The individual biomarkers were pairwise compared between healthy controls, Ward patients, ICU Patients, and Long-COVID patients using a Mann-Whitney U test. A Bonferroni correction was applied to avoid multiple comparison complications, with corrected P-values<0.01 considered to be statistically significant. Individual biomarker boxplots were generated to illustrate the concentration distribution and significance comparison of the biomarker between cohorts.

### Machine Learning:

For machine learning, a Random Forest classifier was used to classify cohorts by their changes in vascular transformation biomarkers. A Random Forest is a set of decision trees and, consequently, we were able to interrogate this collection of trees to identify the features that have the highest predictive value (viz., those features that frequently appear near the top of the decision tree). To reduce overfitting and maintain a conservative model, three-fold cross-validation with a Random Forest of 10 trees and a maximum depth of three was used (25). A Boruta feature reduction algorithm was used to identify the biomarkers with the greatest importance. (26). The common important biomarkers when comparing healthy controls with Long-COVID patients and COVID-19 patients with Long-COVID patients were used to develop a selected biomarker profile.

Receiver operating characteristic (ROC) curves were conducted on the classification results to determine the sensitivity and specificity of individual molecules for predicting Long-COVID status in comparison to healthy controls and COVID-19 patients. Area-under-the-curve (AUC) was calculated as an aggregate measure of protein performance across all possible classification thresholds (27). The biomarker data was visualized with a nonlinear dimensionality reduction on the full data matrix using the t-distributed stochastic nearest neighbor embedding (t-SNE) algorithm. t-SNE assumes that the ‘optimal’ representation of the data lies on a manifold with complex geometry, but a low dimension, embedded in the full-dimensional space of the raw data (28).

A pairwise comparison, using the Euclidian distance, was conducted to determine the similarity between subjects across the selected biomarkers (29). As such, subjects similar across their selected biomarker profile have a lower Euclidian distance compared to subjects with differing biomarker profiles. The distances were visualized using a heatmap of which the scale was adjusted to counter the extremely large distance outliers which reduce the visibility of the other comparisons. Exploratory analysis was also conducted to determine relationships of the selected biomarkers to Long-COVID clinical measures using a Mann-Whitney U test with patients missing clinical data excluded from the analysis. All analysis was conducted using Python version 3.9.7 and Scikit-Learn version 1.0.1.

### Results

A total of 4 age- and sex-matched groups were included consisting of Long-COVID outpatients (median years old=61; IQR=19; n=23), Ward COVID-19 inpatients (median years old=60; IQR=20; n=23), ICU COVID-19 inpatients (median years old=60; IQR=17; n=23) and health control subjects (median years old=59; IQR=16; n=23). There were no significant differences with regards to age (P=0.9869) and sex (P=0.9877) between the 4 cohorts. Baseline demographic characteristics, comorbidities, laboratory measurements, interventions, and chest x-ray findings of Long-COVID outpatients and the Ward/ICU COVID-19 inpatients, are reported in Table 1 and Table 2, respectively. Long-COVID outpatients had a single blood draw at their clinic visit, whereas blood from Ward and ICU COVID-19 inpatients was drawn on day 1 and day 3. Long-COVID patients had significantly elevated lymphocyte measurements in comparison to both Ward and ICU COVID-19 patients as determined by a Kruskal-Wallis H-test (p<0.0001). The mortality rates for Ward and ICU COVID-19 inpatients were 8.7% and 47.8%, respectively.

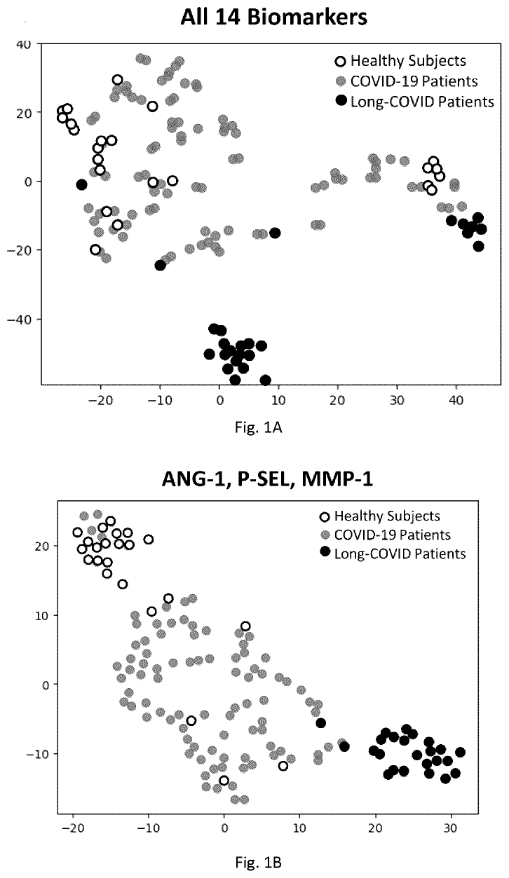

Sixteen vascular transformation blood biomarkers were measured using multiplex immunoassay technology (Table 3), with fourteen being significantly different between cohorts (P<0.01 to P<0.0001; Table 4 and Table 5). Using all 14 vascular transformation blood biomarkers, a t-SNE plot illustrated that Long-COVID patients were easily separable from acutely ill COVID-19 inpatients and healthy control subjects (Table 5, FIG. 1A; classification accuracy 88.2%). A shortlisted biomarker model was created with Boruta feature reduction and included ANG-1, P-Sel and MMP-1. The selected model using only ANG-1, P-Sel and MMP1 produced a t-SNE plot illustrating a separation more distinct between the three cohort groups, with a near-perfect separation of Long-COVID patients (FIG. 1B; classification accuracy 98%).

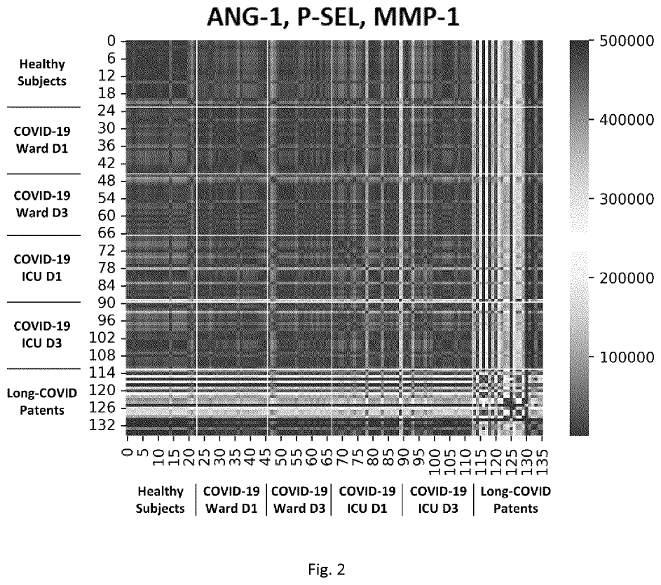

To compare the cohorts in terms of a holistic profile containing the selected 3 biomarkers, ANG-1, P-Sel and MMP1, Euclidean distances between all subjects were calculated and demonstrated distinct biomarker profiles represented by larger distances between subjects (FIG. 2). The biomarker profiles of Healthy Control Subjects, Ward COVID-19 inpatients and ICU COVID-19 inpatients were homogeneous as shown by the low Euclidean distances. Although the biomarker profile of all Long-COVID patients is not homogenous, there was a distinct difference between the biomarker profile of Long-COVID patients and that of the other patient cohorts.

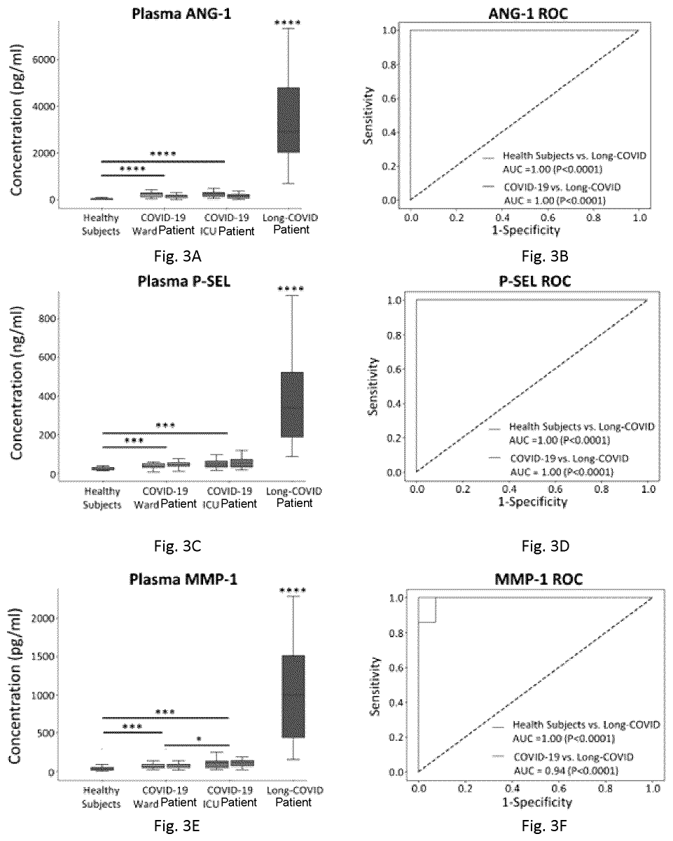

An individual boxplot comparison of the concentrations of the leading three vascular transformation biomarkers, ANG1, P-SEL, and MMP1 illustrated that all were significantly elevated (P<0.0001) in Long-COVID patients as compared to the other cohorts (FIGS. 3A, 3C and 3E). FIGS. 3B, 3D and 3F show ROC curve analyses that demonstrated excellent diagnostic potential for ANG-1 (AUC 1.0; P<0.0001), P-Sel (AUCs 1.0; P<0.0001) and MMP-1 (AUCs 0.94-1.0; P<0.0001). ROC curve analyses for the remaining eleven vascular transformation blood biomarkers are listed in Table 6.

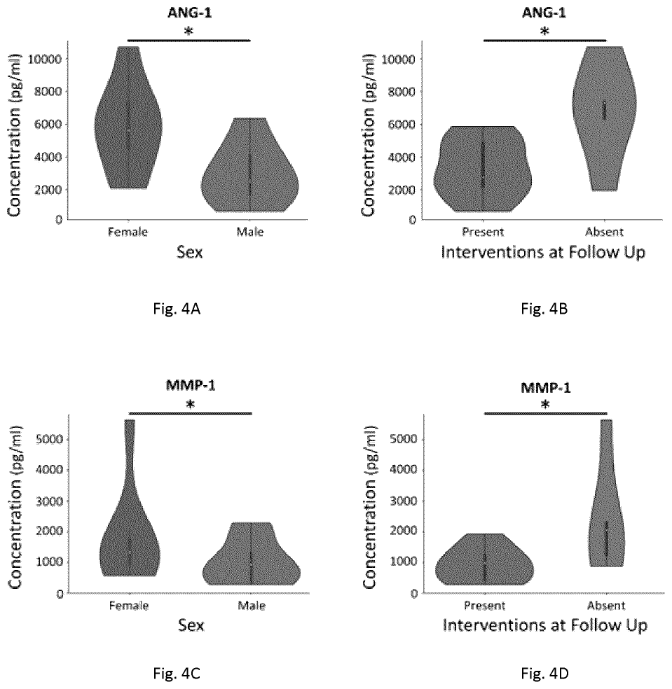

The concentrations of the leading 3 biomarkers, ANG-1, P-Sel and MMP-1, were compared between the demographics and clinical presentations of the Long-COVID outpatients. There were significant differences in both ANG-1 and MMP-1 between females and males (P=0.028 and P=0.038, respectively), with females having a higher median blood concentration (FIGS. 4A and 4C). There were also significant differences in both ANG1 and MMP1 concentrations for those Long-COVID outpatients that had an intervention at the follow-up compared to those that had no intervention (P=0.038 and P=0.038, respectively). Those without an intervention had a higher median concentration of ANG-1 and MMP-1 than those with an intervention at follow-up (FIGS. 4B and 4D). There was no association between those Long-COVID outpatients that had an intervention on follow-up and the sex of the Long-COVID patients as determined by a Fisher Exact Test (P=0.69).

TABLE 1
Long COVID-19 Outpatient Demographics and Clinical Data

| Initial Infection Variable | Outpatients (n = 23) | |
|---|---|---|
| Age (yrs), median (IQR) | 61.0 | (19.0) |
| Male sex, no. (%) | 13 | (56.5) |
| Diagnostic test: PCR, serology, no. (%) | 23 | (100.0) |
| Vaccination status at infection, no. (%) | 2 | (8.7) |
| Hospitalization, no. (%) | | |
| Ward | 7 | (30.4) |
| ICU | 1 | (4.3) |
| Comorbidities, no. (%) | | |
| Diabetes | 6 | (26.1) |
| Hypertension | 8 | (34.8) |
| Coronary artery/heart disease | 2 | (8.7) |
| Chronic/congestive heart failure | 0 | (0.0) |
| Chronic kidney disease | 0 | (0.0) |
| Cancer | 1 | (4.3) |
| COPD | 0 | (0.0) |
| Asthma | 4 | (17.4) |
| Presenting symptoms at infection, no. (%) | | |
| Fever | 16 | (69.6) |
| Cough | 18 | (78.3) |
| Anosmia/Ageusia | 14 | (60.9) |
| Pharyngitis | 9 | (39.1) |
| Headache | 14 | (60.9) |
| Confusion/Memory | 2 | (8.7) |
| Myalgias | 13 | (56.5) |
| Dyspnea | 16 | (69.6) |
| Chest pain | 8 | (34.8) |
| Nausea/Vomiting/Diarrhea | 12 | (52.2) |
| Interventions at infection, no. (%) | | |
| Steroids | 7 | (30.4) |
| Remdesivir | 1 | (4.3) |
| Tocilizumab | 1 | (4.3) |
| Long-COVID Clinic Variables | | |
| Follow up, days from infection onset, median (IQR) | 98.5 | (47.5) |
| Lingering symptoms at follow up, no. (%) | | |
| Respiratory | 16 | (69.6) |
| Cardiovascular | 6 | (26.1) |
| Neurology | 9 | (39.1) |
| Musculoskeletal | 1 | (4.3) |
| Gastro-Intestinal | 3 | (13.0) |
| Psychiatric | 1 | (4.3) |
| Cutaneous | 0 | (0.0) |
| Balance | 0 | (0.0) |
| Chest pain | 4 | (17.4) |
| Concentration | 1 | (4.3) |
| Cough | 2 | (8.7) |
| Dyspnea | 16 | (69.6) |
| Fatigue | 11 | (47.8) |
| Headache | 2 | (8.7) |
| Low mood | 1 | (4.3) |
| Anxiety | 1 | (4.3) |
| Memory | 7 | (30.4) |
| Nausea | 1 | (4.3) |
| Palpitations | 1 | (4.3) |
| Paresthesia | 1 | (4.3) |
| Smell/taste | 2 | (8.7) |
| Word finding | 2 | (8.7) |
| Non-specific | 11 | (47.8) |
| Laboratories at follow up, median (IQR) | | |
| White blood cell count | 7.1 | (2.0) |
| Neutrophils | 4.5 | (1.6) |
| Lymphocytes | 2.0 | (0.6) |
| Hemoglobin | 140.0 | (22.5) |
| Platelets | 230.0 | (59.5) |
| C-Reactive Protein (CRP) | 1.8 | (3.5) |
| Ferritin | 86.5 | (133.8) |
| Lactate Dehydrogenase (LDH) | 201.0 | (37.0) |
| Alanine Aminotransferase (ALT) | 20.0 | (10.5) |
| Interventions at follow up, no. (%) | | |
| Pulmicort | 1 | (4.3) |
| Anticoagulant | 1 | (4.3) |
| Symbicort | 10 | (43.5) |
| Ventolin | 3 | (13.0) |
| Lasix | 1 | (4.3) |
| Nasal spray | 2 | (8.7) |
| Oxygen | 2 | (8.7) |
| Physiotherapy | 5 | (21.7) |
| None | 8 | (34.8) |

TABLE 2
Acutely III COVID-19 Inpatient Demographics and Clinical Data

| | Ward Inpatients | | ICU Inpatients | |
|---|---|---|---|---|
| Variable | (n = 23) | | (n = 23) | |
| Age (yrs), median (IQR) | 60.0 | (20.0) | 60.0 | (17.0) |
| Male sex, no. (%) | 14 | (60.9) | 13 | (56.5) |
| Weight (kg), median (IQR) | 86.0 | (13.4) | 89.8 | (26.5) |
| Height (cm), median (IQR) | 170.0 | (8.0) | 171.0 | (8.5) |
| BMI, median (IQR) | 28.1 | (5.4) | 30.3 | (7.1) |
| MODS, median (IQR) | — | | 5.0 | (1.8) |
| SOFA Score, median (IQR) | — | | 6.0 | (5.5) |
| Comorbidities, no. (%) | | | | |
| Diabetes | 4 | (17.4) | 10 | (43.5) |
| Hypertension | 9 | (39.1) | 10 | (43.5) |
| Coronary artery/heart disease | 1 | (4.3) | 2 | (8.7) |
| Chronic/congestive heart failure | 0 | (0.0) | 0 | (0.0) |
| Chronic kidney disease | 1 | (4.3) | 2 | (8.7) |
| Cancer | 3 | (13.0) | 2 | (8.7) |
| COPD | 0 | (0.0) | 1 | (4.3) |
| Presenting symptoms, no. (%) | | | | |
| Fever | 18 | (78.3) | — | |
| Cough | 19 | (82.6) | — | |
| Anosmia/Ageusia | 5 | (21.7) | — | |
| Pharyngitis | 5 | (21.7) | — | |
| Headache | 3 | (13.0) | — | |
| Myalgias | 14 | (60.9) | — | |
| Dyspnea | 20 | (87.0) | — | |
| Chest pain | 3 | (13.0) | — | |
| Nausea/Vomiting/Diarrhea | 10 | (43.5) | — | |
| Pulmonary pathology, no. (%) | | | | |
| Unilateral pneumonia | — | | 1 | (4.3) |
| Bilateral pneumonia | 22 | (95.7) | 21 | (91.3) |
| Interstitial infiltrates/R effusion | — | | 1 | (4.3) |
| Laboratories, median (IQR) | | | | |
| Hemoglobin | 130.0 | (24.0) | 119.0 | (28.5) |
| White Blood Cell count | 7.0 | (5.0) | 8.8 | (7.5) |
| Neutrophils | 5.9 | (4.2) | 7.6 | (6.9) |
| Lymphocytes | 0.8 | (0.6) | 0.7 | (0.6) |
| Platelets | 219.0 | (80.5) | 216.0 | (131.0) |
| Creatinine | 70.0 | (28.5) | 81.0 | (113.5) |
| International Normalized Ratio | 1.1 | (0.1) | 1.2 | (0.1) |
| Lactate | 1.7 | (1.0) | 1.3 | (0.8) |
| Partial thromboplastin time (PTT) | — | | 27.0 | (5.0) |
| PaO₂/FiO₂ Ratio | — | | 128.0 | (70.0) |
| Interventions, no. (%) | | | | |
| Renal replacement therapy | 0 | (0.0) | 6 | (26.1) |
| High-flow nasal cannula | 13 | (56.5) | 15 | (65.2) |
| Non-invasive mechanical ventilation | 1 | (4.3) | 7 | (30.4) |
| Invasive mechanical ventilation | 2 | (8.7) | 21 | (91.3) |
| Extracorporeal membrane | | | | |
| oxygenation | 0 | (0.0) | 1 | (4.3) |
| Tocilizumab | 2 | (8.7) | 0 | (0.0) |
| Steroids | 22 | (95.7) | 15 | (65.2) |
| Vasoactive medications | 2 | (9.5) | 19 | (82.6) |
| Antibiotics | 22 | (95.7) | 23 | (100.0) |
| Anti-virals | 5 | (21.7) | 3 | (13.0) |
| Antiplatelet | 4 | (17.4) | 18 | (78.3) |
| Anticoagulation | 23 | (100.0) | 22 | (95.6) |
| Outcomes | | | | |
| Days, median (IQR) | 8.0 | (7.0) | 15.0 | (14.0) |
| Died, no. (%) | 2 | (8.7) | 11 | (47.8) |

TABLE 3
Function of 16 Vascular Transformation Biomarkers

| | Biomarker | Function |
|---|---|---|
| 1 | ANG-1 | Angiopoietin-1 (ANG-1), part of the |
| | | angiopoietin family, expressed on endothelial |
| | | cells, induces TIE2 tyrosine kinase and activates |
| | | angiogenesis and vascular protective effects |
| | | including suppressing plasma leakage, inhibiting |
| | | vascular inflammation, preventing endothelial |
| | | death, and enlargement of existing vessels. |
| 2 | P-SEL | P-Selectin (P-SEL), expressed on endothelial |
| | | cells and platelets, facilitates platelet |
| | | aggregation, platelet adhesion, endothelial cell |
| | | migration, mononuclear cell proliferation. |
| 3 | MMP-1 | Matrix Metalloproteinases 1 (MMP-1) cleaves |
| | | peptide bonds of the extracellular matrix |
| | | during the initial angiogenesis and wound |
| | | healing stages. Over, unregulated expression |
| | | can lead to harmful processes including tumor- |
| | | promoting proliferation, invasion, and metastasis. |
| 4 | VE-Cad | Vascular Endothelial Cadherin (VE-Cad) is a |
| | | cell-cell adhesion glycoprotein and critical to |
| | | maintaining a restrictive endothelial barrier. |
| | | Blocking VE-Cadherin inhibits angiogenesis and |
| | | the formation of vascular structures in vitro. |
| 5 | Syn-1 | Syndecan-1 (Syn-1), is expressed primarily on |
| | | epithelial cells but also endothelial cells and |
| | | leukocytes. Syn-1 binds ligands, including |
| | | ICAM-1 and VCAM-1, via its Heparan sulfate |
| | | chain. Syn-1 in inflammation negatively |
| | | regulates leukocyte adhesion and migration. |
| 6 | Endoglin | Auxiliary receptor for the transforming growth |
| | | factor-β (TGF- β). Elevated expression |
| | | during inflammation and wound healing, is |
| | | critical for angiogenesis and maintaining |
| | | normal vascular architecture. |
| 7 | PECAM-1 | Platelet endothelial cell adhesion molecule-1 |
| | | (PECAM-1) is a cell-cell adhesion molecule on |
| | | vascular cells including platelets, endothelial |
| | | cells, lymphocytes, and is involved in leukocyte |
| | | transmigration, signaling, angiogenesis. |
| 8 | VEGF-A | Vascular endothelial growth factor A (VEGF-A) |
| | | generated by hypoxia-increased VEGF gene |
| | | transcription, resulting in angiogenesis |
| | | processes: cell migration, proliferation, |
| | | microvascular permeability, and MMP activity. |
| 9 | ICAM-1 | Increased Intercellular adhesion molecule-1 |
| | | (ICAM-1) expression in endothelial cells, |
| | | macrophages, and lymphocytes by proinflamma- |
| | | tory cytokines (IL-1, TNF). Facilitates |
| | | leukocyte LFA-1 binding to endothelial cells |
| | | and enables leukocyte transmigration. |
| 10 | VLA-4 | Very Late Antigen-4 (Integrin α4β1, |
| | | VLA-4), part of integrins family of immune |
| | | cell adhesion receptors where VLA-4 specifically |
| | | facilitates leukocyte arrest on endothelium and |
| | | transmigration during inflammation. Expression |
| | | regulated by growth factors or chemokines, |
| | | common ligand is VCAM-4 and fibronectin. |
| 11 | E-SEL | E-selectin (E-SEL) is expressed by endothelial |
| | | cells when upregulated by inflammatory cytokines |
| | | (TNF-α and IL-1β) and binds carbohydrate |
| | | ligands expressed by leukocytes to facilitate |
| | | recruitment. |
| 12 | Thrombomodulin | A transmembrane glycoprotein expressed on |
| | | endothelial, immune, lung alveolar epithelial |
| | | cells. Decreases intravascular coagulation by |
| | | 1. Binding and sequestering thrombin (converts |
| | | fibrinogen and activates platelets) 2. |
| | | Thrombin-thrombomodulin pathway |
| | | downregulates thrombin generation. |
| 13 | VEGF-R2 | Vascular endothelial growth factor receptor-2 |
| | | (VEGF-R2) in vascular endothelial cells |
| | | facilitates VEGF-A, -C, and -D binding and |
| | | signals for angiogenesis and vasculogenic |
| | | activation. VEGF-R2 also regulates endothelial |
| | | cell growth, differentiation, and migration. |
| 14 | VEGF-R3 | Vascular endothelial growth factor receptor-3 |
| | | (VEGF-R3) primarily in lymphatic endothelial |
| | | cells binds VEGF-C and -D and signals for |
| | | lymphangiogenesis. VEGF-R3 is also implicated |
| | | in regulating angiogenesis and vascular |
| | | network formation via VEGF-A. |
| 15 | VEGF-D | Vascular endothelial growth factor D (VEGF-D), |
| | | expressed in all tissues but predominantly in |
| | | lung and skin, binds VEGF-R2 and VEGF-R3 to |
| | | increase angiogenesis and lymphangiogenesis. |
| | | Unprocessed VEGF-D has a poor affinity to |
| | | VEGF-R2, proteolytic processed VEGF-D |
| | | increases affinity to VEGF-R2 and boosts the |
| | | affinity of VEGF-R3. |
| 16 | VCAM-1 | Vascular cell adhesion protein 1 (VCAM-1) is |
| | | part of the immunoglobulin family, expressed in |
| | | endothelial cells and functions as a ligand for |
| | | VLA-4, and regulates leukocyte transmigration. |

TABLE 4
Pair-Wise Comparisons of Sixteen vascular transformation blood biomarkers.

| | Long- | Long- | Long- | | | |
|---|---|---|---|---|---|---|
| | COVID vs. | COVID vs. | COVID vs. | Healthy | Healthy | Ward vs. |
| Biomarker | Healthy | Ward | ICU | vs. ICU | vs Ward | ICU |
| ANG-1 | <0.0001 | <0.0001 | <0.0001 | <0.0001 | <0.0001 | 1 |
| P-SEL | <0.0001 | <0.0001 | <0.0001 | 0.0001 | 0.0004 | 1 |
| MMP-1 | <0.0001 | <0.0001 | <0.0001 | <0.0001 | 0.0005 | 0.0437 |
| VE-Cad | <0.0001 | <0.0001 | <0.0001 | 0.0014 | 0.0024 | 1 |
| Syn-1 | 0.0013 | 1 | 0.1485 | <0.0001 | 0.0003 | 0.0002 |
| Endoglin | 0.0047 | <0.0001 | <0.0001 | 0.0138 | 0.0112 | 1 |
| PECAM-1 | <0.0001 | <0.0001 | <0.0001 | 1 | 0.4165 | 0.7330 |
| VEGF-A | <0.0001 | <0.0001 | 0.0015 | <0.0001 | 0.0044 | 0.0034 |
| ICAM-1 | 0.0016 | 0.0600 | 0.0347 | 0.0048 | 0.1105 | 1 |
| VLA-4 | <0.0001 | <0.0001 | <0.0001 | 0.8992 | 1 | 1 |
| E-SEL | <0.0001 | <0.0001 | <0.0001 | 1 | 0.0037 | <0.0001 |
| Thrombomodulin | 0.0005 | <0.0001 | 0.0156 | 0.1742 | 1 | 0.0196 |
| VEGF-R2 | <0.0001 | 0.0002 | 0.002 | 0.2953 | 1 | 1 |
| VEGF-R3 | 0.0006 | 0.0004 | <0.0001 | 1 | 1 | 1 |
| VCAM-1 | 0.0683 | 0.3346 | 1 | 0.1580 | 1 | 0.2305 |
| VEGF-D | 1 | 0.1037 | 0.5067 | 0.5073 | 0.0890 | 1 |

Long-COVID, Long-COVID outpatients (n = 23); Healthy, health control subjects (n = 23); Ward, acutely ill Ward inpatients (n = 23); ICU, acutely ill ICU inpatients (n = 23).

TABLE 5
Relative importance of fourteen blood biomarkers
for classifying subjects between cohorts
Feature Ranking

| | Analyte | % Importance |
|---|---|---|
| 1 | ANG-1 | 21.7 |
| 2 | P-SEL | 15.4 |
| 3 | MMP-1 | 12.5 |
| 4 | VE-Cad | 9.3 |
| 5 | Syn-1 | 7.8 |
| 6 | Endoglin | 6.0 |
| 7 | PECAM-1 | 5.4 |
| 8 | VEGF-A | 5.4 |
| 9 | ICAM-1 | 5.3 |
| 10 | VLA-4 | 3.3 |
| 11 | E-SEL | 3.1 |
| 12 | Thrombomodulin | 1.8 |
| 13 | VEGF-R2 | 1.7 |
| 14 | VEGF-R3 | 1.5 |

TABLE 6
Classification accuracy (Random Forest Classifier) and Area Under the Curve (AUC:
ROC Curve Analyses) of Sixteen vascular transformation blood biomarkers.

| | Long-COVID vs. Healthy Control | | Long-COVID vs. COVID-19 | |
|---|---|---|---|---|
| | Classification | | Classification | |
| | Accuracy | | Accuracy | |
| Biomarker | (%) | AUC | (%) | AUC |
| ANG-1 | 98 | 1.00 | 97 | 1.00 |
| P-SEL | 98 | 1.00 | 94 | 1.00 |
| MMP-1 | 97 | 1.00 | 91 | 0.94 |
| VE-Cad | 78 | 0.91 | 84 | 0.96 |
| Syn-1 | 69 | 0.79 | 76 | 0.47 |
| Endoglin | 74 | 0.79 | 78 | 0.88 |
| PECAM-1 | 85 | 0.98 | 86 | 0.96 |
| VEGF-A | 87 | 0.96 | 81 | 0.83 |
| ICAM-1 | 87 | 0.81 | 78 | 0.71 |
| VLA-4 | 70 | 0.86 | 85 | 0.85 |
| E-SEL | 76 | 0.91 | 86 | 0.89 |
| Thrombomodulin | 78 | 0.84 | 80 | 0.82 |
| VEGF-R2 | 82 | 0.92 | 76 | 0.77 |
| VEGF-R3 | 74 | 0.81 | 79 | 0.79 |
| VCAM-1 | 56 | 0.74 | 81 | 0.56 |
| VEGF-D | 41 | 0.52 | 75 | 0.35 |

In this study, we measured 16 vascular transformation blood biomarkers obtained from age- and sex-matched Long-COVID outpatients, Ward COVID-19 inpatients, ICU COVID-19 inpatients and healthy control subjects. To identify the leading biomarkers distinguishing Long-COVID patients from the other cohorts, we utilized both conventional statistics and state-of-the-art machine learning. Our data indicate a unique Long-COVID blood proteome; three vascular transformation biomarkers were identified that distinguished Long-COVID patients from acutely ill COVID-19 inpatients and healthy control subjects (classification accuracy of 98%). Our data suggest that one or more of the three leading biomarkers, ANG-1, P-Sel and MMP-1, can be used as disease biomarkers. Moreover, given the primary roles of these three biomarkers in angiogenesis, accelerators or inhibitors of microvascular re-modelling, ANG-1, P-Sel and MMP-1 provide therapeutic use for Long-COVID patients.

Our patient cohorts were similar to those reported in earlier studies with regards to demographics, comorbidities and clinical presentation. For example, Long-COVID patients were more likely to be older, have greater body mass index and be female sex (30). They suffered diffuse symptoms, such as fatigue, post-exertional malaise, anosmia and cognitive dysfunction, and across multiple organ systems (14, 31). With regards to acutely ill COVID-19 patients, they were similar to those reported in earlier cohorts (32-35), and as per multiple studies, suffered significant inflammatory and thrombotic mechanisms (5, 6, 36, 37), associated with microvascular injury (9). Our study has identified 14 vascular transformation proteins that are significantly elevated in Long-COVID outpatients, with the leading three proteins (ANG-1, P-Sel and MMP-1), taken alone or in any possible combination (i.e., ANG-1, P-Sel, MMP-1, ANG-1/P-Sel, ANG-1/MMP-1, P-Sel/MMP-1, and ANG-1/P-Sel/MMP-1) accurately identifying Long-COVID status. The strength of the leading three proteins for identifying Long-COVID status was demonstrated with both conventional statistics as well as state-of-the-art machine learning techniques. We also provide a rank order listing of the importance of these three molecules relative to the full set of significantly different molecules, resulting in a limited biomarker profile for efficient clinical translatability. The molecules are critical for vascular transformation and suggest they play a wound-healing role in the Long-COVID patients. Information regarding the function of the other molecules is provided in the supplementary documents.

Angiopoietin 1 (ANG-1), part of the angiopoietin family, is a secreted glycoprotein ligand that induces the tyrosine phosphorylation of TIE2 tyrosine kinase expressed exclusively in endothelial cells (38). Previous studies have shown ANG-1 to have vasculature protective effects including suppressing plasma leakage, inhibiting vascular inflammation, preventing endothelial death, and enlargement of existing vessels (39, 40). The Angiopoietin-TIE2 pathway defends against acute or chronic lung injury and disruptions in the pathway may lead to deterioration in microvascular integrity (41). ANG-2, an Angiopoietin-TIE2 antagonist, disrupts the vascular barrier and its elevation is associated with COVID-19 ICU mortality (42). As COVID-19 patients have increased angiogenesis due to the endothelial injury (10), the significantly elevated ANG-1 observed in our Long-COVID patients may represent a long-term, wound-repairing angiogenesis response.

P-Sel (P-Selectin) is a type-1 transmembrane glycoprotein that is expressed on endothelial cells and platelets (43). During infection, the endothelium is activated and P-Sel facilitates platelet aggregation and adhesion (44, 45). P-Sel also serves a critical role angiogenesis by promoting early inflammatory mononuclear cell proliferation (46), and mediating endothelial cell migration (47). P-Sel is acutely elevated in COVID-19, and it is associated with COVID-19 symptom severity (48-51). Lymphocyte binding along specialized high endothelial venules is initiated by P-Sel (52), and may be associated with our observation of elevated lymphocytes in Long-COVID outpatients, relative to acutely ill COVID-19 inpatients. We could not establish an association between P-Sel and platelet number, suggesting the role of P-Sel in Long-COVID may be skewed towards angiogenesis and lymphocyte migration, rather than platelet aggregation.

MMP-1 is a part of a family of zinc matrix metalloproteinases that cleave peptide bonds of the extracellular matrix (ECM) proteins including collagen types I-III, laminins, elastin, and fibronectin (53, 54). Breakdown of the ECM occurs during the initial stages of angiogenesis and wound healing to allow for new cell proliferation and migration (55). MMP-1 is critical for angiogenesis in vitro due to its endothelial remodeling abilities (53), and MMP-1 activates protease-activated-receptor-1 (PAR-1) which plays an important role in angiogenesis (56). MMP-1 in Long-COVID may contribute to increased angiogenesis and wound healing, as well as, platelet aggregation (55).

Accumulating evidence suggests that females have a greater chance of being affected by Long-COVID (30, 57). Given the sex- and age-matched nature of our study, the Long-COVID group has roughly an equal number of males (56.5%) and females (43.5%), and thus we cannot corroborate those findings directly. However, ANG-1 and MMP-1 were both significantly elevated in our female Long-COVID patients, as opposed to males. Some aspects of these findings should be treated with caution as ANG-1 is generally higher in females than males (58) due to the essential role of angiogenesis during fetal development and the female reproductive cycle (59).

Our study has also identified ANG-1 and MMP-1 to be significantly elevated in Long-COVID individuals receiving no interventions at follow-up. There were no sex differences for individuals receiving interventions and those without interventions. These latter findings suggest that ANG-1 and MMP-1, two proteins that are critically important for angiogenesis, are protective at higher levels, perhaps by hastening the healing response.

Our study has identified 14 significantly elevated vascular transformation biomarkers and developed a three-protein profile for Long-COVID status. Our study investigated 4 patient cohorts and we ensured robust analysis via non-parametric statistics and conservative machine learning techniques. Our data show elevated biomarkers in Long-COVID patients at 3-5 months after acute infection and our results demonstrate a potential for one or more vascular transformation biomarkers to identify Long-COVID status The development of diffuse symptoms and biomarker changes after a PCR-positive acute illness, or in tandem with SARS-CoV-2 nucleocapsid antibody testing, would support Long-COVID diagnosis.

### REFERENCES

1. Harrison A G, Lin T, Wang P. Mechanisms of SARS-CoV-2 Transmission and Pathogenesis. Trends in Immunology. 2020; 41(12):1100-15.
2. Yuki K, Fujiogi M, Koutsogiannaki S. COVID-19 pathophysiology: A review. Clinical Immunology. 2020; 215:108427.
3. Koyama S, Ishii K J, Coban C, Akira S. Innate immune response to viral infection. Cytokine. 2008; 43(3):336-41.
4. Garvin M R, Alvarez C, Miller J I, Prates E T, Walker A M, Amos B K, et al. A mechanistic model and therapeutic interventions for COVID-19 involving a RAS-mediated bradykinin storm. Elife. 2020; 9.
5. Fraser D D, Cepinskas G, Slessarev M, Martin C, Daley M, Miller M R, et al. Inflammation Profiling of Critically Ill Coronavirus Disease 2019 Patients. Crit Care Explor. 2020; 2(6):e0144.
6. Gill S E, dos Santos C C, O'Gorman D B, Carter D E, Patterson E K, Slessarev M, et al. Transcriptional profiling of leukocytes in critically ill COVID19 patients: implications for interferon response and coagulation. Intensive Care Medicine Experimental. 2020; 8(1):75.
7. Juneja G K, Castelo M, Yeh C H, Cerroni S E, Hansen B E, Chessum J E, et al. Biomarkers of coagulation, endothelial function and fibrinolysis in critically-ill patients with COVID-19: A single-centre prospective longitudinal study. J Thromb Haemost. 2021.
8. Cani E, Dwivedi D J, Liaw K-L, Fraser D D, Yeh C H, Martin C, et al. Immunothrombosis Biomarkers for Distinguishing Coronavirus Disease 2019 Patients From Noncoronavirus Disease Septic Patients With Pneumonia and for Predicting ICU Mortality. Critical Care Explorations. 2021; 3(12).
9. Fraser D D, Patterson E K, Slessarev M, Gill S E, Martin C, Daley M, et al. Endothelial Injury and Glycocalyx Degradation in Critically Ill Coronavirus Disease 2019 Patients: Implications for Microvascular Platelet Aggregation. Crit Care Explor. 2020; 2(9):e0194.
10. Ackermann M, Verleden S E, Kuehnel M, Haverich A, Welte T, Laenger F, et al. Pulmonary Vascular Endothelialitis, Thrombosis, and Angiogenesis in Covid-19. N Engl J Med. 2020.
11. Bertsimas D, Lukin G, Mingardi L, Nohadani O, Orfanoudaki A, Stellato B, et al. COVID-19 mortality risk assessment: An international multi-center study. PLOS ONE. 2020; 15(12):e0243262.
12. Raveendran A V, Jayadevan R, Sashidharan S. Long COVID: An overview. Diabetes & Metabolic Syndrome: Clinical Research & Reviews. 2021; 15(3):869-75.
13. Crook H, Raza S, Nowell J, Young M, Edison P. Long covid-mechanisms, risk factors, and management. BMJ. 2021:n1648.
14. Davis H E, Assaf G S, McCorkell L, Wei H, Low R J, Re'Em Y, et al. Characterizing long COVID in an international cohort: 7 months of symptoms and their impact. EClinicalMedicine. 2021; 38:101019.
15. Huang C, Huang L, Wang Y, Li X, Ren L, Gu X, et al. 6-month consequences of COVID-19 in patients discharged from hospital: a cohort study. The Lancet. 2021; 397(10270):220-32.
16. Salamanna F, Maglio M, Landini M P, Fini M. Body Localization of ACE-2: On the Trail of the Keyhole of SARS-CoV-2. Front Med (Lausanne). 2020; 7:594495.
17. Gavriilaki E, Eftychidis I, Papassotiriou I. Update on endothelial dysfunction in COVID-19: severe disease, long COVID-19 and pediatric characteristics. Journal of Laboratory Medicine. 2021; 45(6):293-302.
18. Matta J, Wiernik E, Robineau O, Carrat F, Touvier M, Severi G, et al. Association of Self-reported COVID-19 Infection and SARS-CoV-2 Serology Test Results With Persistent Physical Symptoms Among French Adults During the COVID-19 Pandemic. JAMA Internal Medicine. 2022; 182(1):19-25.
19. Prevention CfDCa. CDC 2019-Novel Coronavirus (2019-nCoV) Real-Time RT-PCR Diagnostic Panel 2021 [Available from: https://www.fda.gov/media/13492/download.
20. NIH HRPP. POLICY: Guidelines for Limits of Blood Drawn for Research Purposes in the Clinical Center. M95-9 (rev). 2009; June 5.
21. Brisson A R, Matsui D, Rieder M J, Fraser D D. Translational research in pediatrics: tissue sampling and biobanking. Pediatrics. 2012; 129(1):153-62.
22. Gillio-Meina C, Cepinskas G, Cecchini E L, Fraser D D. Translational research in pediatrics II: blood collection, processing, shipping, and storage. Pediatrics. 2013; 131(4):754-66.
23. Priestap F, Kao R, Martin C M. External validation of a prognostic model for intensive care unit mortality: a retrospective study using the Ontario Critical Care Information System. Can J Anaesth. 2020.
24. Singer M, Deutschman C S, Seymour C W, Shankar-Hari M, Annane D, Bauer M, et al. The Third International Consensus Definitions for Sepsis and Septic Shock (Sepsis-3). JAMA. 2016; 315(8):801-10.
25. Tang C, Garreau D, von Luxburg U, editors. When do random forests fail?NeurIPS; 2018.
26. Kursa M B, Rudnicki W R. Feature selection with the Boruta package. J Stat Softw. 2010; 36(11):1-13.
27. Bradley A P. The use of the area under the ROC curve in the evaluation of machine learning algorithms. Pattern recognition. 1997; 30(7):1145-59.
28. Van der Maaten L, Hinton G. Visualizing data using t-SNE. Journal of machine learning research. 2008; 9(11).
29. Jambu M. Chapter 10—Classification of Individuals-Variables Data Sets. In: Jambu M, editor. Exploratory and Multivariate Data Analysis. Boston: Academic Press; 1991. p. 305-405.
30. Sudre C H, Murray B, Varsavsky T, Graham M S, Penfold R S, Bowyer R C, et al. Attributes and predictors of long COVID. Nature Medicine. 2021; 27(4):626-31.
31. Carfi A, Bernabei R, Landi F. Persistent Symptoms in Patients After Acute COVID-19. JAMA. 2020; 324(6):603.
32. Myers L C, Parodi S M, Escobar G J, Liu V X. Characteristics of Hospitalized Adults With COVID-19 in an Integrated Health Care System in California. JAMA. 2020; 323(21):2195.
33. Bhatraju P K, Ghassemieh B J, Nichols M, Kim R, Jerome K R, Nalla A K, et al. Covid-19 in Critically Ill Patients in the Seattle Region—Case Series. N Engl J Med. 2020.
34. Zhou F, Yu T, Du R, Fan G, Liu Y, Liu Z, et al. Clinical course and risk factors for mortality of adult inpatients with COVID-19 in Wuhan, China: a retrospective cohort study. Lancet. 2020; 395(10229):1054-62.
35. Wu C, Chen X, Cai Y, Xia J A, Zhou X, Xu S, et al. Risk Factors Associated With Acute Respiratory Distress Syndrome and Death in Patients With Coronavirus Disease 2019 Pneumonia in Wuhan, China. JAMA Internal Medicine. 2020; 180(7):934.
36. Fraser D D, Cepinskas G, Patterson E K, Slessarev M, Martin C, Daley M, et al. Novel Outcome Biomarkers Identified With Targeted Proteomic Analyses of Plasma From Critically Ill Coronavirus Disease 2019 Patients. Crit Care Explor. 2020; 2(9):e0189.
37. Fraser D D, Slessarev M, Martin C M, Daley M, Patel M A, Miller M R, et al. Metabolomics Profiling of Critically Ill Coronavirus Disease 2019 Patients: Identification of Diagnostic and Prognostic Biomarkers. Crit Care Explor. 2020; 2(10):e0272.
38. Davis S, Aldrich T H, Jones P F, Acheson A, Compton D L, Jain V, et al. Isolation of Angiopoietin-1, a Ligand for the TIE2 Receptor, by Secretion-Trap Expression Cloning. Cell. 1996; 87(7):1161-9.
39. Brindle N P J, Saharinen P, Alitalo K. Signaling and Functions of Angiopoietin-1 in Vascular Protection. Circulation Research. 2006; 98(8):1014-23.
40. Thurston G. Complementary actions of VEGF and Angiopoietin-1 on blood vessel growth and leakage*. Journal of Anatomy. 2002; 200(6):575-80.
41. Sack K D, Kellum J A, Parikh S M. The Angiopoietin-Tie2 Pathway in Critical Illness. Crit Care Clin. 2020; 36(2):201-16.
42. Vassiliou A G, Keskinidou C, Jahaj E, Gallos P, Dimopoulou I, Kotanidou A, et al. ICU Admission Levels of Endothelial Biomarkers as Predictors of Mortality in Critically Ill COVID-19 Patients. Cells. 2021; 10(1):186.
43. Tvaroska I, Selvaraj C, Koca J. Selectins—The Two Dr. Jekyll and Mr. Hyde Faces of Adhesion Molecules-A Review. Molecules. 2020; 25(12):2835.
44. Furie B, Furie B C. Role of platelet P-selectin and microparticle PSGL-1 in thrombus formation. Trends Mol Med. 2004; 10(4):171-8.
45. Lorant D E, Topham M K, Whatley R E, Mcever R P, Mcintyre T M, Prescott S M, et al. Inflammatory roles of P-selectin. Journal of Clinical Investigation. 1993; 92(2):559-70.
46. Egami K, Murohara T, Aoki M, Matsuishi T. Ischemia-induced angiogenesis: role of inflammatory response mediated by P-selectin. Journal of Leukocyte Biology. 2006; 79(5):971-6.
47. Morbidelli L, Brogelli L, Granger H J, Ziche M. Endothelial cell migration is induced by soluble P-selectin. Life Sci. 1998; 62(1):P17-11.
48. Fraser D D, Patterson E K, Slessarev M, Gill S E, Martin C, Daley M, et al. Endothelial Injury and Glycocalyx Degradation in Critically Ill Coronavirus Disease 2019 Patients: Implications for Microvascular Platelet Aggregation. Crit Care Explor. 2020; 2(9):e0194-e.
49. Venter C, Bezuidenhout J A, Laubscher G J, Lourens P J, Steenkamp J, Kell D B, et al. Erythrocyte, Platelet, Serum Ferritin, and P-Selectin Pathophysiology Implicated in Severe Hypercoagulation and Vascular Complications in COVID-19. International Journal of Molecular Sciences. 2020; 21(21):8234.
50. Goshua G, Pine A B, Meizlish M L, Chang C H, Zhang H, Bahel P, et al. Endotheliopathy in COVID-19-associated coagulopathy: evidence from a single-centre, cross-sectional study. The Lancet Haematology. 2020; 7(8):e575-e82.
51. Yatim N, Boussier J, Chocron R, Hadjadj J, Philippe A, Gendron N, et al. Platelet activation in critically ill COVID-19 patients. Annals of Intensive Care. 2021; 11(1).
52. Diacovo T G, Puri K D, Warnock R A, Springer T A, Von Andrian U H. Platelet-mediated lymphocyte delivery to high endothelial venules. Science. 1996; 273(5272):252-5.
53. Fisher C, Gilbertson-Beadling S, Powers E A, Petzold G, Poorman R, Mitchell M A. Interstitial Collagenase Is Required for Angiogenesis in Vitro. Developmental Biology. 1994; 162(2):499-510.
54. Unemori E N, Ferrara N, Bauer E A, Amento E P. Vascular endothelial growth factor induces interstitial collagenase expression in human endothelial cells. Journal of cellular physiology. 1992; 153(3):557-62.
55. Sang Q X A. Complex role of matrix metalloproteinases in angiogenesis. Cell Research. 1998; 8(3):171-7.
56. Tressel S L, Kaneider N C, Kasuda S, Foley C, Koukos G, Austin K, et al. A matrix metalloprotease-PAR1 system regulates vascular integrity, systemic inflammation and death in sepsis. EMBO Molecular Medicine. 2011; 3(7):370-84.
57. Bai F, Tomasoni D, Falcinella C, Barbanotti D, Castoldi R, Mule G, et al. Female gender is associated with long COVID syndrome: a prospective cohort study. Clinical Microbiology and Infection. 2021.
58. Sullivan J C, Rodriguez-Miguelez P, Zimmerman M A, Harris R A. Differences in angiotensin (1-7) between men and women. American journal of physiology Heart and circulatory physiology. 2015; 308(9):H1171-H6.
59. Gupta K. Angiogenesis: a curse or cure?Postgraduate Medical Journal. 2005; 81(954):236-42.

Through the embodiments that are illustrated and described, the currently contemplated best mode of making and using the disclosure is described. Without further elaboration, it is believed that one of ordinary skill in the art can, based on the description presented herein, utilize the present disclosure to the full extent. All publications cited herein are incorporated by reference.

Although the description above contains many specificities, these should not be construed as limiting the scope of the disclosure, but as merely providing illustrations of some of the presently embodiments of this disclosure.

1. A method of diagnosing lonq-COVID in a subject, the method comprising: (a) measuring the level of one or more biomarkers in a test sample obtained from the subject, and (b) comparing the level of the one or more biomarkers in the test sample with a healthy control and/or an acute COVID-19 reference level value of said one or more biomarkers, wherein an increase in the level of the one or more biomarkers in the test sample relative to the healthy control and/or acute COVID-19 reference level value of said one or more biomarkers is indicative of lonq-COVID diagnosis, and wherein the one or more biomarkers are selected from ANG-1, P-Sel, MMP-1, VE-Cad, Syn-1, Endoqlin, PECAM, VEGF-A, ICAM-1, VLA-4, E-Sel, Thrombomodulin, VEGF-R2, and VEGF-R-3, wherein when the subject is diagnosed with lonq-COVID-19, the method further includes treating the subject with a lonq-COVID therapy.

2. The method of claim 1, wherein the one or more biomarkers include one or more of ANG-1, P-Sel and MMP-1.

3. The method of claim 1, wherein the one or more biomarkers is ANG-1. P-Sel or MMP-1.

4. The method of claim 1, wherein the one or more biomarkers are ANG-1 and P-Sel.

5. The method of claim 1, wherein the one or more biomarkers are ANG-1 and MMP-1.

6. The method of claim 1, wherein the one or more biomarkers are P-Sel and MMP-1.

7. The method of claim 1, wherein said one or more assays is a proteomic assay.

8. (canceled)

9. The method of claim 1, wherein said therapy include one or more interventions listed in Table 1.

10. The method of claim 1, wherein said therapy comprises administering to the subject a treatment that promotes angiogenesis.

11. The method of claim 1, wherein said therapy comprises administering to the subject accelerators of angiogenesis.

12. The method of claim 1, wherein said therapy comprises administering to the subject one or more of the biomarkers listed in Table 5.

13. The method of claim 1, wherein said therapy comprises administering to the subject one or more of ANG-1, P-Sel and MMP-1.

14. A method of treating lonq-COVID in a subject, the method comprising administering to the subject one or more of ANG-1, P-Sel, MMP-1, VE-Cad, Syn-1, Endoglin, PECAM, VEGF-A, ICAM-1, VLA-4, E-Sel, Thrombomodulin, VEGF-R2, and VEGF-R-3.

15. The method of claim 14, wherein the one or more biomarkers are ANG-1, P-Sel and MMP-1.

16. A method of treating lonq-COVID subject, the method comprising providing a subject having an increase in the level of one or more biomarkers relative to a healthy control and/or an acute COVID-19 reference level value of said one or more biomarkers, and (b) treating the subject with a therapy effective against lonq-COVID, wherein the one or more biomarkers include ANG-1, P-Sel, MMP-1, VE-Cad, Syn-1, Endoqlin, PECAM, VEGF-A, ICAM-1, VLA-4, E-Sel, Thrombomodulin, VEGF-R2, and VEGF-R-3.

17. The method of claim 16, wherein said therapy includes one or more interventions listed in Table 1.

18. The method of claim 16, wherein said therapy comprises administering to the subject a treatment that promotes angiogenesis.

19. The method of claim 16, wherein said therapy comprises administering to the subject accelerators of angiogenesis.

20. The method of claim 16, wherein said therapy comprises administering to the subject one or more of the biomarkers.

21. The method of claim 16, wherein said therapy comprises administering to the subject one or more of ANG-1, P-Sel and MMP-1.

22-25. (canceled)

26. The method of claim 1, wherein step (b) is done using machine learning.

27. A method of diagnosing long-COVID in a subject, the method comprising: (a) using machine learning to compare the level of one or more biomarkers in a test sample obtained from the subject with healthy control and/or acute COVID-19 reference level value of said one or more biomarkers, wherein the one or more biomarkers are selected from ANG-1, P-Sel, MMP-1, VE-Cad, Syn-1, Endoglin, PECAM, VEGF-A, ICAM-1, VLA-4, E-Sel, Thrombomodulin, VEGF-R2, and VEGF-R-3, and (b) determining whether the subject is positive or negative for long-COVID based on said comparison.
